(19) United States
(12) Patent Application Publication
(10) Pub. No.: US 2004/0047776 A1
(43) Pub. Date: Mar. 11, 2004

(54) Mobile air decontamination method and device

(76) Inventor: James M. Thomsen, Key West, FL (US)

Correspondence Address: JAMES M. THOMSEN, BOX 5572, KEY WEST, FL, 33045, US

(21) Appl. No.: 10/442,031

(22) Filed: May 20, 2003

Related U.S. Application Data
(60) Provisional application No. 60/382,126, filed on May 20, 2002.

(51) Int. Cl. B32B 5/02; B01J 19/08

(52) U.S. Cl. 422/186.07; 422/121; 422/186.3

(57) ABSTRACT

Air decontamination method and device designed for bioterrorism, nerve gas, toxic mold, small pox, Ebola, anthrax and other agents require built in air sampling, rapid filter changes and the ability to use a mobile, transportable and connectable system in positive mode to push contaminates away or in negative mode to contain a toxin from spreading. This application combines features in respirators, industrial and hospital grade air filtration with the ability to provide air testing to guide the connection of the device with other treatment modules or existing HVAC and other equipment. With this new flexibility, ozone, UV, absorption, Thermal destruction, filters and liquid chemical neutralization can be manually or automatically adapted for emergency response to both daily airborne contamination and military grade terrorist threats of airborne contamination. The air decontamination units may be used to decontaminate the air after industrial and medical contaminations and terrorist biological, chemical and radiological attacks, for example. Mobile isolation units, and methods of decontaminating rooms, are disclosed, as well as Well as infection control and emergency response usage as an emergency clean air supply when connected to escape hoods, decon tents, or containment barriers to protect structures from homes to business from outside toxic agents. The unit can be powered by normal AC, 120 volts or 240 or be adapted to battery or field power supply units.

The present application claims the benefit under 35 U.S.C. § 119(e) of U.S. Provisional Patent Application No. 60/382,126 filed on May 20, 2003, which is incorporated by reference, herein.

Air Decontamination Devices and Methods that uses six possible air treatment modules or treatments in multiple, adaptable, stages in a high volume mobile system.

More particularly, germicidal air filters, decontamination devices and mobile isolation units including new methods for combining different treatment sections either manually or automatically to respond to air borne contamination determined from built in air sampling data collection systems

Air decontaminate devices differ from simple Air filtration devices because of the volume, capacity and efficiency required for toxic levels of contaminated atmosphere where safety and health is threatened. Air Filtration devices treat an anticipated level of contamination without the ability to sample, define toxins, change configurations of air treatment and track effectiveness.

### BACKGROUND OF THE INVENTION

The last five years in the united states has present airborne risks of unprecedented nature at the release of multiple toxins at the World Trade Center, Anthrax attacks, SARS and Smallpox concerns, and other newly developing toxic air borne concerns such as toxic mold.

While industrial air filtration devices have been made in the past for asbestos, laboratory and hospital use they have been inefficient in capturing and controlling the toxic airborne plume because they are designed to be fixed or attached systems and the plumes are moving, expanding and changing based on a wide range of environmental and other factors.

Home air cleaners do not contain the high volume capability or capacity to address the overwhelming assault of toxic mold or agents used by terrorists or other more concentrated hazards such as smallpox and SARS.

Previously, air cleaners were self contained and designed to function only with their components and parts and could not be connected to existing HVAC systems, rooms, functional spaces in a flexible by effective configuration.

Previously air decontamination devices were not designed to allow connection to other air treatment technology devices such as a fume hood exhaust or modules to treat special hazards by heat, gas, absorption, and liquid neutralization.

Air Decontamination devices have typically been designed to filter, irradiate, and/or trap irritants or infectious agents, such as bacteria, viruses, mold and other microorganisms, chemicals, and particulate, in air. Such irritants and infectious agents may contaminate the air due to industrial accidents, fires, an infected individual, or a chemical or biological terrorist attack, for example. Existing methods and devices frequently use either filters or trapping methods for particulates and for gases, chemical or biological agents they might add or use UV light, heat, absorption, chemical treatments or create chambers where mixtures of gas are applied to neutralize the airborne hazard. Biological decontamination air filter devices typically comprise a chamber to expose contaminated air to ultraviolet (“UV”) radiation followed by a filter. The filter may be a high efficiency particle arrester (“HEPA”) filter.

Most air high volume industrial air movement air filter and treatment devices are “fixed” or attached systems that are mounted as part of a HVAC or other collection system and are not mobile. This includes fume hoods and air treatment devices that are mounted on ceilings, walls, floors and as part of existing air movement systems that move over 500 CFM of air per minute. Smaller volume air treatment or filtration systems are typically portable or mobile such as a vacuum cleaner with attached HEPA air filter which move liters instead of meters of air per minute. The need to have portable, high volume, high capacity air treatment systems with a variety of mobile configurations is acute because rather than bring the contaminated air to the treatment device, the treatment device needs to come to the contaminated air and move with the dynamic cloud or source of the toxin. This creates less risk to humans and other life. A similar development in respirators occurred when it was found having ventilated areas was not enough and that portable protective filter respirators were needed to more fully protect workers exposed to toxins as they moved from areas of high or low, or safe and non safe air borne contamination.

Most air treatment devices could only address airborne toxins in one category at a time. These were solids, liquids, gasses or biological contamination. Filters are the most common air treatment option, with chemical reaction or absorbents for liquids and gases and occasionally gas treatment use as ozone. The inability for mobile devices to be manually configured with choices of filters, gases, UV light, heat, and chemical reactions has left the nation unprepared to respond to many emerging threats of bioterrorism.

Air treatment systems such as filters, do not typically offer any way to identify and take samples of the airborne contaminates in the air stream the treat which is important to guide the operator in choosing the best technology option to remove the toxic threat. Documenting levels of pre treatment and post treatment contaminates has been used for asbestos and environmental cleanup projects but the sampling is done with separate devices, separate staffs and typically take days for results to be available to adjust the air treatment technology.

Ultraviolet irradiation in prior art devices is typically unable to sufficiently penetrate the filters to kill trapped biological agents. Many biological agents, such as mold and bacteria, can grow on moist filter media. The filter media, including such mold and bacteria, as well as trapped viruses, may thereby become a source of contamination and infection. Since some deadly viruses and bacteria can survive for extended periods of time in filters, removal of the contaminated filters may release the very contaminant the decontamination unit was intended to contain. For example, they can cause infection of a person replacing the filter or conducting maintenance on the decontamination device. They may also become a source of infection of people in a room with the device.

In many of these devices, ultraviolet irradiation alone may not provide sufficient decontamination because the contaminated air is not exposed to the radiation for a sufficient time to kill the biological agents. High energy ultraviolet irradiation, such as ultraviolet germicidal irradiation in the wavelength range of 2250-3020 Angstroms(“UVGI”), has been used to irradiate filters but UVGI alone may still not adequately destroy biological agents caught within the filter because in the prior art configurations, the biological agents are not exposed to UVGI irradiation for a sufficient time, and the UVGI irradiation may not adequately penetrate the filter.

U.S. Pat. No. 5,330,722 to Pick et al. (“Pick”) provides a UV lamp to expose a surface of a filter to UV irradiation, as the UV lamp and filter are moved with respect to each other. The UV lamp is only exposed to a portion of the filter at any given time. This design may not allow for an adequate germicidal effect upon agents that may pass through portions of the filter that are displaced with respect to the UV lamp. Although Pick suggests providing a UV lamp that is also capable of producing germicidal levels of ozone that can pass through the filter, the ozone and UV are still unable to destroy agents passing through portions of the filter that are not exposed to the UV lamp. Since agents passing through the filter are returned to the air, filtration of the air may be inadequate.

To improve the germicidal effect in a filter, filters have been coated with germicidal agents. For example, in U.S. Pat. No. 5,766,455 to Berman et al., the filter is coated with metal oxide catalysts that are activated by UV light to degrade chemicals and biological agents. Because this requires modifying filters with a metal oxide catalyst slurry, the filters have added expense and require an additional step of quality control to verify that the dynamics of the filter, such as size of particles trapped and maximum air flow, have not been altered.

Isolation rooms, isolation chambers and isolation areas in hospitals, laboratories and manufacturing facilities may filter contaminated or potentially contaminated air and vent the filtered air to a safe area for dilution. As above, the filters may become dangerous sources of infection and have to be collected and disposed of accordingly. Mobile isolation units are also known, enabling the expansion of isolation zones in hospitals to facilitate the handling of diseased patients, for example. However, mobile isolation units draw significant amounts of air into the unit, potentially exposing patients to further infection. Since antibiotic resistant strains of bacteria and fungus may be present in hospitals, these isolation units may be dangerous to immune or respiratory compromised patients.

Improved decontamination units and isolation devices are needed to better address typical contamination situations in industrial and medical applications, for example, as well as increasingly dangerous threats posed by antibiotic resistant strains and terrorism.

Emergency use decontamination devices need to provide ports to create negative vacuum pressure or gas or liquid neutralization to reduce hazards from captured toxins within the system. These are not available presently in mobile response systems.

Emergency use air decontamination devices should provide emergency provision of breathing air for trapped victims or operators with failure of the personal protective equipment by way of a rear attachment port to provide clean, treated, and filtered air to hoods or tents. These are not available presently in mobile response systems

These units should be mobile, connectable to other systems, and have the ability to connect to other treatment technology or modules as needed or indicated by real time active air sampling.

### SUMMARY OF THE INVENTION

In accordance with an embodiment of the invention, a decontamination device is disclosed comprising a housing defining an air inlet, an air outlet and a path for air to flow from the inlet to the outlet. A stationary filter is positioned within the housing, along the path. The filter has an upstream side to receive air flowing along the path and a downstream side for the exit of air from the filter, to the path. At least one first stationary ultraviolet (“UV”) lamp is positioned to directly illuminate the filter and at least one second stationary UV lamp is positioned to produce Ozone gas when desired on the downstream side of the filter. An ozone generator is proximate the filter. By providing direct UV illumination of glass fiber filters, the light is carried by the glass fibers to glow all parts of the filter in UV light., the UV radiation has greater overall penetration of the filter, enabling the killing of biological agents trapped within or traversing the filter. It is believed that the filter slows the motion of the biological agents, giving the UV radiation more time to act on the agents. In addition, providing the ozone generator on the downside of the filter allows for ozone to permeate the filter, providing another mechanism for killing biological agents in the filter because Ozone has twice the germicidal effect of bleach. The filter may comprise material that is transmissive to ultraviolet radiation, facilitating penetration of the filter by the radiation. The filter thereby becomes an enhanced killing zone. The filter may be sterilized instead of becoming a source of contamination, as in the prior art.

A blower may be provided within the housing, along the path, to cause air to flow along the path during operation. The ultraviolet lamps may completely illuminate the upstream and downstream sides of the filter, respectively. This may further enhance the effectiveness of the UV radiation on and in the filter.

The device is both mobile and high volume converting the power of a roof top HVAC unit into a portable wheel barrel sized package.

The device can be connected to 120 or 240 volts AC or use a battery inverter for power.

The device can connect to rooms, spaces, tents, plastic barriers, existing HVAC units, and push or pull air flows to create positive or negative pressure to spaces or areas to control the airflow of toxic air away from potential victims. The connections can be soft or hard ducts, tunnels or pipes.

The device can be connected to various pre or post treatment modules that add absorption, gas treatment, chemical neutralization, heat or cold treatment, and various specialty filters.

A small air sampling vacuum pump is placed downsteam of the filters to create a suction applied and transmitted to tubes. Three plastic tubes create a suction draw to place sampling at the entry of the unit before air is treated, at the exhaust of the unit to test air so treated and at the operators level. At least one air sampling port is provided through a wall of the housing of the decontamination unit, to provide communication from an exterior of the housing to the path. The air in the vicinity of the decontamination unit may thereby be drawn through a sampling device in the port, for testing of the air to identify contaminants.

At least one prefilter may be positioned along the path, upstream of the main filter ultraviolet lamp, such that air flows through the at least one prefilter prior to flowing through the filter, during operation. The prefilter may provide filtration of gases, as well as biological and chemical contaminants, depending on the type of prefilter. The prefilter may be selected based on testing of the contaminated air. The type of prefilter may be selected based on the results of air sampling.

The filter may be a V-bank filter to allow the UV lamp may be partially within the V-shaped regions defined by the filter, to further improve the irradiation of the filter by the UV lamps.

The device has movable wheels with locking front wheels, a top and back handle that allow the unit to stand on end or be moved quickly by any average adult.

The device as an underside cleanout port connection to allow attachment of suction for a HEPA vacuum, or to serve as an injection port for gas or liquids to neutralize captured toxins before maintenance or filter changes are performed.

The device as a rapid change drop in slot for prefilter selection which and hold one or two prefilters of varying density, composition and thickness.

The device can be set up to push clean air into a functional space or pull contaminated air away from a functional space in a push or pull format and be connected to HVAC systems or connect to other air treatment devices or modules.

The device can act as a high volume ozone generator to push ozone into a functional space for disinfection or other purposes

In accordance with an aspect of this embodiment, a method of decontaminating air is disclosed comprising flowing air through a filter having an upstream side receiving air to be filtered and a downstream side from which filtered air exits the filter. The method further comprises can saturate the filter and air with ozone while the air is flowing through the filter.

In accordance with another embodiment of the invention, a decontamination unit is disclosed comprising a housing defining an inlet, an outlet that allows a duct or connection to other units or treatment modules to align the most efficient decontamination of the air. These modules may be connected together in a caboose like fashion or connected by ducts. The treatment modules can include but are not limited to absorption materials such as treated or untreated organic or synthetic fibers. Charcoal, zeonite, baking soda and other absorbent treatments. Another module can contain heating elements for thermal destruction or cooling elements for condensation. Another module can contain a mist or wet membrane treatment with would apply buffer or reactive solutions to neutralize harmful chemical agents. Finally a module for the introduction of gas such as carbon dioxide, or chlorine dioxide as well as oxygen and others can also be assembled to this air treatment train. The path of the air though the modules will vary by operator selection based on air sampling. A filter is positioned along the path to filter air flowing along the path to remove particulates. The filter comprises a plurality of transverse intersecting walls defining at least one upstream facing chamber to receive air along the path, and a downstream side for air to exit from the filter, to the path. At least one ultraviolet lamp is provided upstream of the filter, facing the at least one chamber, to completely, directly illuminate at least one chamber. A blower may be provided within the housing, along the path, to cause air to flow along the path during operation.

The method may also further comprise permeating the filter with ozone while the air is flowing through the filter.

In accordance with another embodiment of the invention, a the decontamination unit has rapid change filter slots aligned to allow for three second filter changes while the system is still running. This rapid change filter slot allows the filter selection to be altered as conditions warrant without losing the dilution, UV and other treatments.

In accordance with another embodiment of the invention, a decontamination unit is disclosed comprising a housing defining an inlet, an outlet, and a path for air to flow from the inlet to the outlet. A filter is positioned along the path, to filter air flowing along the path. The housing has an external wall defining an air sampling port through the wall, enabling communication between an exterior of the housing and the path. A blower may be provided within the housing, along the path, to move air from the inlet to the outlet. The blower may be downstream of the filter. The port may be an air sampling port and air may be drawn from the exterior of the housing, through the port, to the path. A sampling tube or a particulate collector may be provided in a port to collect air. A selectable prefilter may be provided along the path, upstream of the filter. The selectable filter may be selected based on air sampling results.

In accordance with an aspect of this embodiment, a method of decontaminating air with a decontamination unit is disclosed comprising flowing air along a path through the unit. The path includes a filter and the air is filtered. The method further comprises collecting an air sample, via the unit. The air sample may be of air external to the unit. A prefilter may be selected based on sampling results, and positioned upstream of the filter in the decontamination unit.

In accordance with another embodiment of the invention, a method of decontaminating a room is disclosed comprising producing germicidal concentrations of ozone throughout the room, causing air in the room to flow through a filter, from an upstream side of the filter to a downstream side of the filter and illuminating the upstream and downstream sides of the filter with germicidal levels of ultraviolet light.

In accordance with another embodiment of the invention, a method of decontaminating a room is disclosed comprising drawing air from the room through a filter having an upstream side to receive the air and a downstream side for air to exit the filter and illuminating the filter with ultraviolet light, while the air is flowing through the filter. The entire downstream side of the filter is also illuminated with ultraviolet light and the filter is permeated with ozone while the air is flowing through the filter. The filtered air is ducted out of the room if it is treated with ozone to create a negative pressure within the room. The room may be a prison cell, for example.

In accordance with another embodiment of the invention, a method of decontaminating a room is disclosed comprising flowing air outside of the room through a filter having an upstream side to receive the air and a downstream side from which the air exits the filter. The entire upstream side and downstream side of the filter are illuminated with ultraviolet light and the filter is permeated with ozone while the air is flowing through the filter. The filtered air is ducted into the room to create a positive pressure within the room.

### BRIEF DESCRIPTION OF THE DRAWINGS

FIG. 1 is a schematic diagram showing the outside side view of a decontamination unit in accordance with an embodiment of the invention;

FIG. 2 is a side view, cross sectional schematic diagram of the decontamination unit of FIG. 1;

FIG. 3 is a top view, cross sectional schematic diagram of the decontamination unit of FIG. 1;

FIG. 4 is an example of a control panel and control circuit that may be used to control operation of the decontamination unit of FIG. 1;

FIG. 5 is a cross sectional schematic diagram of a portion of the housing of the decontamination unit of FIG. 1, showing sampling ports and typical sampling cassettes and tubes attached to these ports for passive or active air sampling;

FIG. 6 is a schematic representation external components and parts of an embodiment of the decontamination unit of FIG. 1;

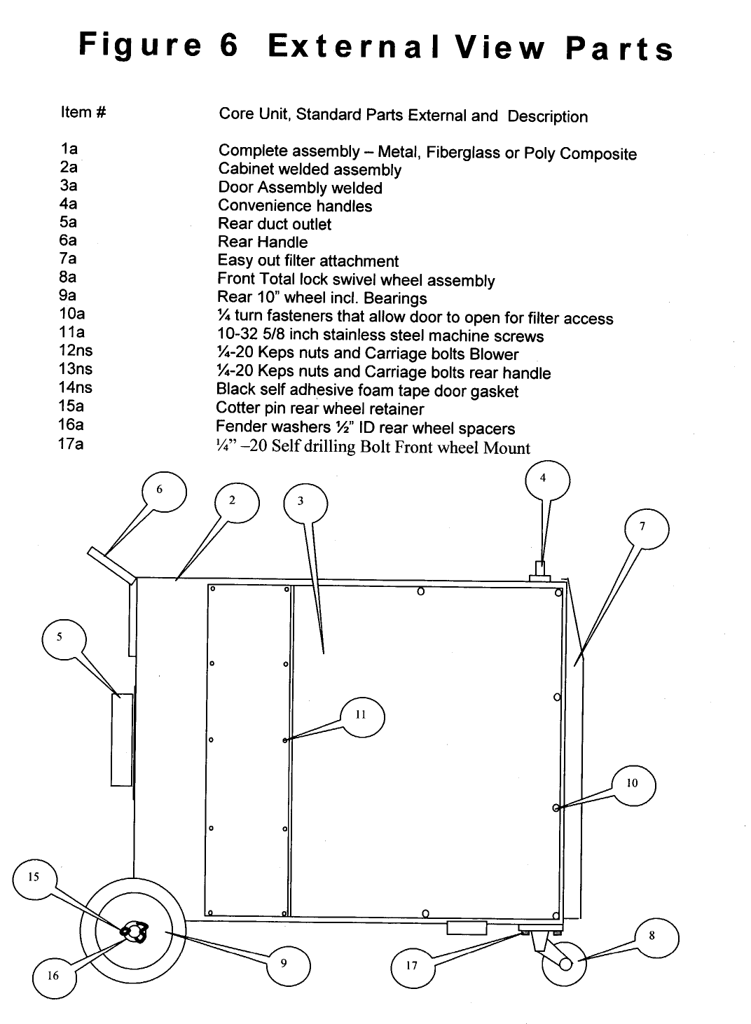

FIG. 8 is representational diagram of the decontamination unit of FIG. 1 used in either horizontal and vertical configurations to more efficiently collect toxic air contamination which may be lighter than air or heaver than air.

FIG. 9; is a schematic diagram representing the ability to attach soft or hard ducts from the intake or output of the decontamination in FIG. 1 to tents, rooms, spaces, and other devices and equipment.

FIG. 10 is a cross sectional schematic diagram of the UV light components that can be installed before, or after filters or treatment areas to provide biological sanitization or creation of disinfectant ozone gas in the decontamination unit of FIG. 1;

FIG. 11 is a cross sectional schematic diagram of the decontamination unit of FIG. 1, in a positive pressure application;

FIG. 12 is a cross sectional schematic diagram of the decontamination unit of FIG. 1, in a negative pressure with one embodiment showing the attachments of funneling plastic curtains to increase efficiency of capture of airborne contamination;

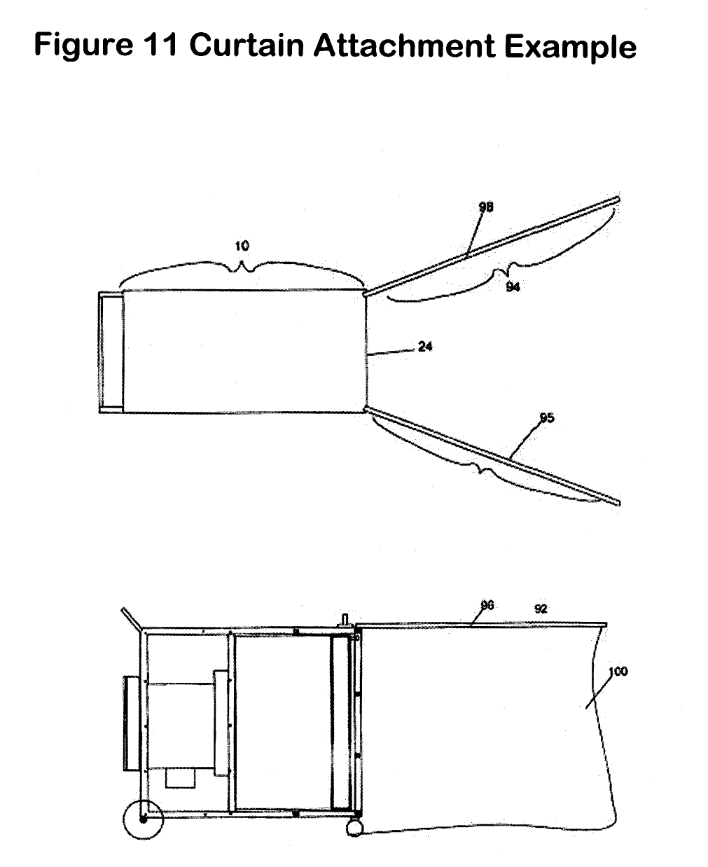

FIG. 12 shows attachment of the decontamination unit using a duct or caboose attachment collars to other air treatment methods such as HEGA module High Efficiency Gas Absorber) Thermal Treatment, or mixing with neutralizing gases, liquids, mists and absorbent treatments;

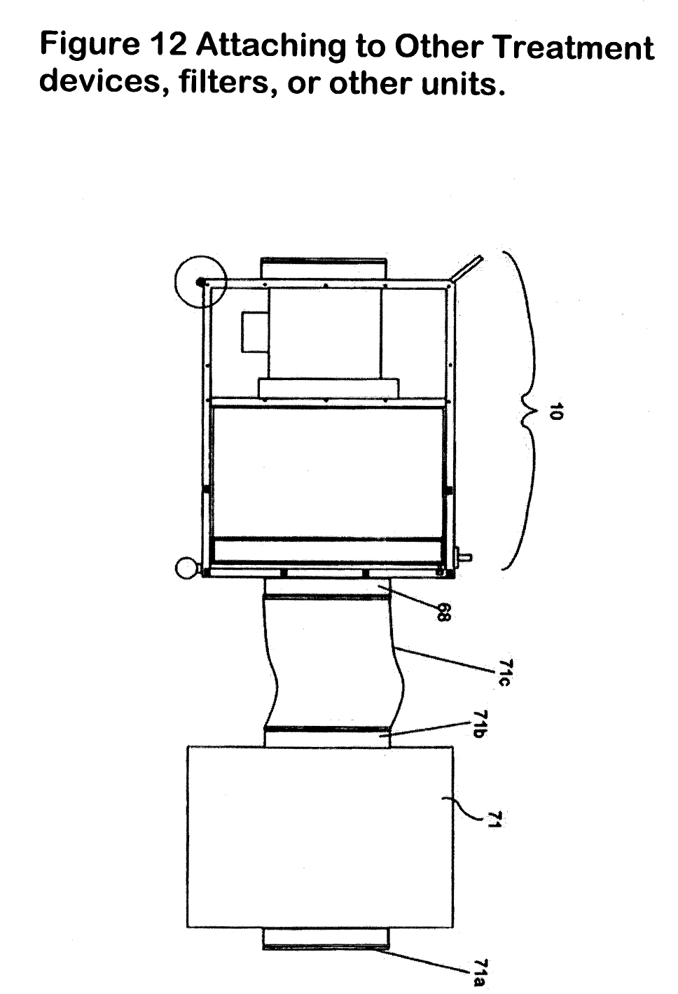

FIG. 13 show decontamination units as in FIG. 1, being used to create negative pressure by sucking contaminated air outward from within a functional space, tent, decon, room, or other area, in accordance with another embodiment of the invention;

FIG. 14 show decontamination units as in FIG. 1, being used to create a pushing air flow movement of clean safe air thus creating positive pressure within a functional space, tent, decon, room, or other area, in accordance with another embodiment of the invention;

FIG. 15 show decontamination units as in FIG. 1, being used to create a “room respirator” and protecting trapped victims by pushing air toxins away towards another area while diluting the toxin by ventilation.

### DETAILED DESCRIPTION OF THE PREFERRED EMBODIMENTS

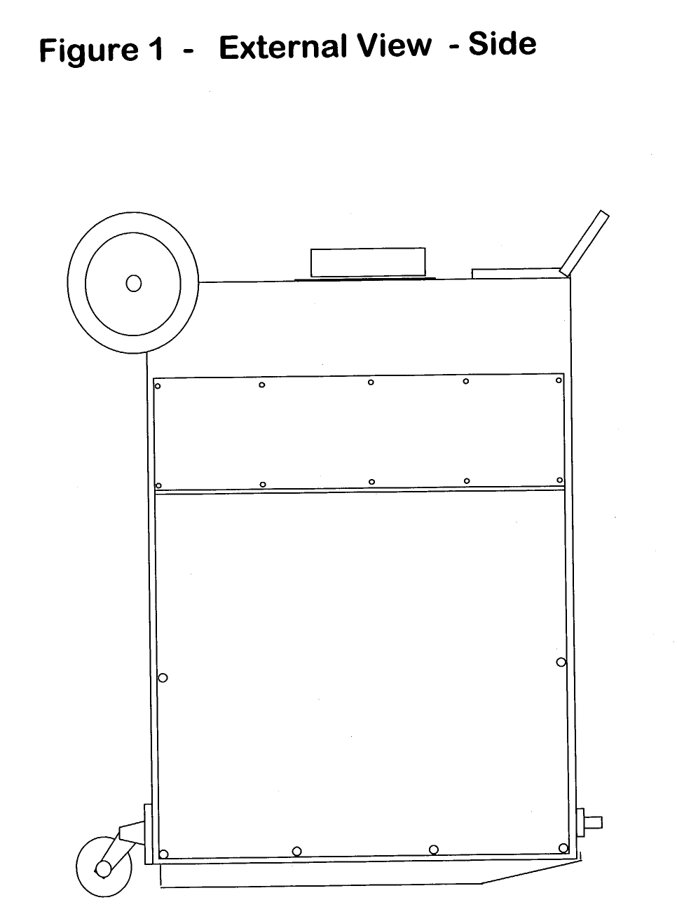

FIG. 1 FIG. 1 is a schematic diagram showing the outside side view of a decontamination unit in accordance with an embodiment of the invention which is designed to be mobile, easily picked up and moved by way of handles and wheels, and is narrow enough to fit down the aisle of commercial aircraft. The unit is made of metal, or plastic and fiberglass and can fit in a standard sized equipment storage area on most fire trucks and emergency vehicles.

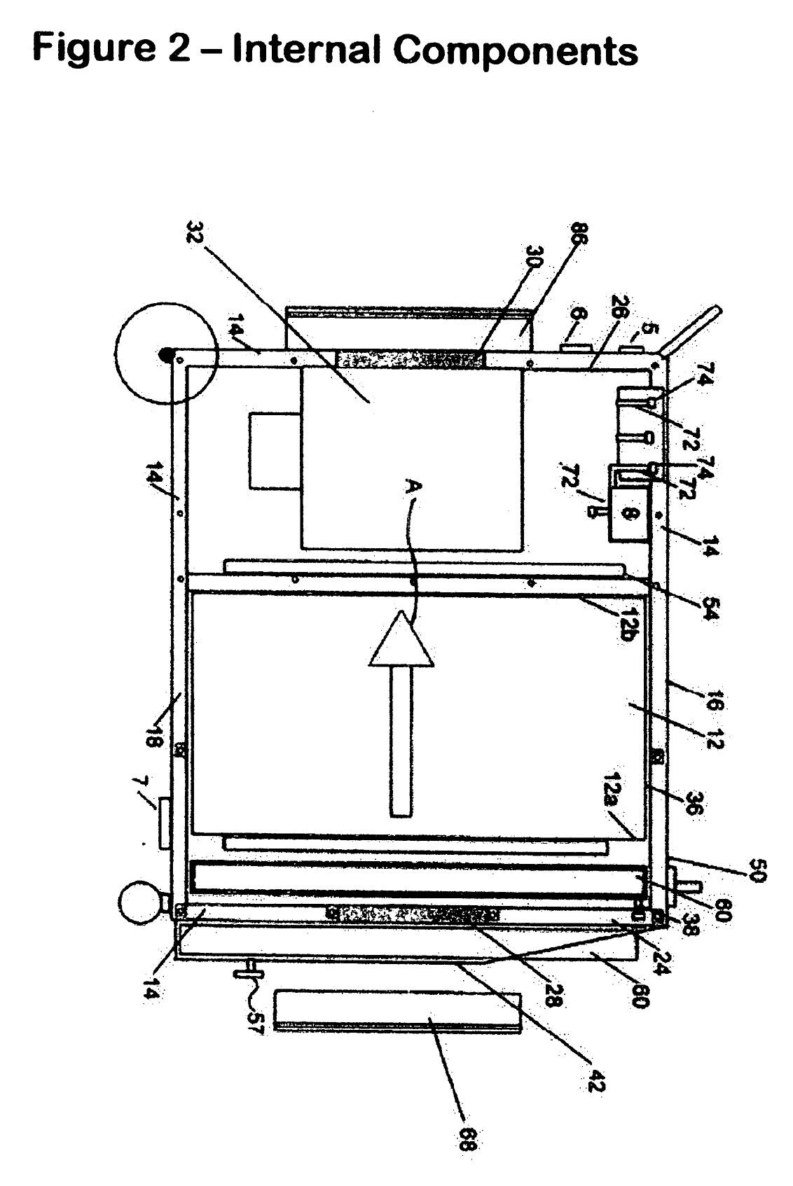

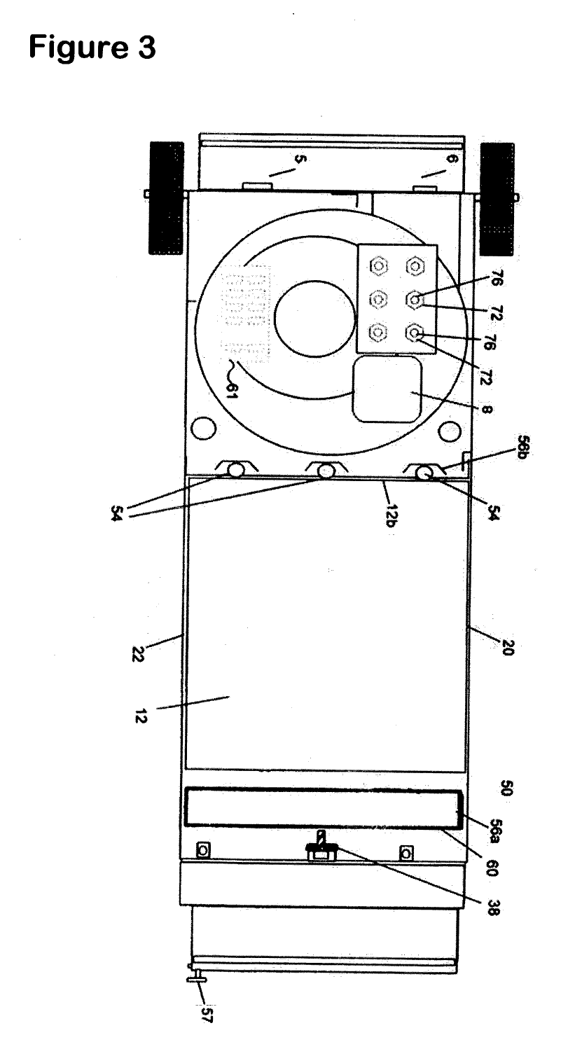

FIG. 2 is representation of a decontamination unit 10 including a filter 12, in accordance with an embodiment of the invention. FIG. 2 is a top cross sectional schematic view of the decontamination unit 10 of FIG. 1. The decontamination unit 10 comprises a housing 14 with a top wall 16, a bottom wall 18, two side walls 20 and 22, a front wall 24 and a back wall 26. An air inlet 28 and an air outlet 30 are defined in the housing 14, in this example in the front wall 24 and the back wall 26. The air inlet 28 and/or the air outlet 30 may be defined in other walls, instead. The housing 14 and structures within the housing define an air path A between the inlet 28 and the outlet 30. The housing 14 is preferably air tight, except for the air inlet 28, the air outlet 30, and passive or active air sampling ports 72 discussed further, below. The walls of the housing 14 are plastic where caustic chemicals are expected for preferably steel for non emergency use. At least one wall should be removable or hinged to facilitate opening so that elements inside of the housing 14 can be maintained. A connection port 7 is located on the bottom to allow attachment of HEPA vacuums or gas sterilization agents to make internal filter changing safe by ensuring all toxins are either contained for made inert. Another connector for a respirator hose 6 at least 1 inch in diameter is located on the output side of the unit to supply a hood with clean air for operator protection should their respiratory protection fail A small electric vacuum pump is also mounted inside 8, to allow air sampling inside the output of the side of the filter to overcome the wind and air flow rates that do not allow for passive sampling Two of the six sampling ports above are connected to this vacuum pump to allow faster sample draw times and the attachment of a vinyl tube taped to the front of the unit for another sampling. This allows the operator to sample the air for toxins at the intake, exhaust and operator level with commercially available sampling cassettes. In this embodiment, a blower 32 is fixed inside of the housing 14, along the air path A, to draw air into the air inlet 28 path A and to discharge air out of the air outlet 30. A blower 32 is a device for pushing or pulling air. Examples of blowers 32 include, but are not limited to, fans and centrifugal blowers. The blower 32 can be fixed to the housing 14 by standard fasteners such as brackets and bolts or machine screws, for example. The blower 32 preferably has multiple or variable speeds. Preferably, operation of the blower 32 is separately controlled by a switch or dial 34, or other such manually operated control device on the housing surface, as shown in FIG. 3. The blower 32 may be outside of the housing 14, coupled to the air outlet 30, to draw air along path A, as well.

The filter 12 is fixed within the housing 14, along the path A so that the air flowing from the air inlet 28 to the air outlet 30 must pass through the filter 12. The blower 24 may be upstream or downstream of the filter 12 to either push or pull air through the filter. Pulling air through the filter 12 is preferred because cleaner (filtered) air causes less wear on the blower 32 during operation. Preferably, the filter 12 is fixed in a manner that prevents air leakage around the filter, yet allows for removal of the filter during replacement. The filter may be fitted tightly within the housing 14, for example. If the filter 12 does not fit tightly within the housing 14, leakage around the filter may be reduced by a flange welded or fixed to the inside of the housing and extending to the filter 12. A compression clamp or tension screw 38 may be used to fix the filter 12 in place, while allowing for easy removal, for example.

One or more ultraviolet (“UV”) lamps 54 are fixed to the housing 14 (or supporting structure within the housing 14). UV lamps 54 are positioned to directly illuminate the glass fibered filter 12, which receives air to be filtered along the air path A. Optionally lamps can be installed on the entire upstream side of the filter 12 is illuminated. One or more UV lamps 54 are also preferably fixed to the housing 14 (or supporting structure within the housing 14), positioned to directly illuminate a downstream side 12b of the filter 12. Filtered air exits the filter 12 from the downstream side 12b.

The ultraviolet lamps 50, preferably provide ultraviolet germicidal irradiation (“UVGI”) 54 at germicidal levels at the filter surfaces 12a, 12b. UVGI is in a range of from about 2250 to about 3020 Angstroms for air/surface disinfection and sterilization.

Concentration of UV germicidal irradiation (UVGI) 54 upon the surface of the filter 12 by reflectors 56 improves the germicidal effect of the UVGI in the filter 12. Examples of germicidal UV lamps include, but are not limited to Perkin Elmer Model GX018T5VH / Ultra-V, Perkin Elmer Optoelectronics, Salem, Mass., USA. The ultraviolet lamps 54 and/or reflectors may be supported by the housing of the decontamination unit 10, as well.

Preferably, filter 12 is a high efficiency filter. In the present invention, a high efficiency filter traps at least 90% of particles of 0.3 microns. More preferably, the high efficiency filter 12 is a high efficiency particle arresting (“HEPA”) filter that traps 99.97% of particles at 0.3 microns, 1000 cubic feet per minute (“CFM”) (28.32 cubic meters per minute). Most preferably, the filter 12 is an ultra high efficiency particulate arresting (“ULPA”) filter that can trap 99.99% of particles at 0.1 microns, at 600-2400 CFM (16.99-67.96 cubic meters per minute). The filter 12 is also preferably fire resistant. Preferably, the fire resistant material is fiberglass, such as a fiberglass mesh, which is also translucent to ultraviolet (“UV”) light. Transmission of the UV light into and through the filter 12 is thereby facilitated. UV light passing into and through the fiberglass mesh irradiates pathogens trapped inside of the mesh of the filter 12. The filter 12 used in the embodiments of this invention does not require coating with photopromoted catalysts, although such catalysts may be used if desired.

The Camfil Farr Filtra 2000^{(™)} Model No. FA 1560-01-01 may be used in the decontamination unit 10 with an airflow of 2,000 CFM (56.63 cubic meters per minute), for example. This model filter has a rated airflow of 2400 CFM (67.96 cubic meters per minute). The dimensions and resistance at airflow of the filter are the same as that of the filter for the Camfil Farr Filtra 2000^{™} Model No. FA 1565-01-01filter rated at 900 CFM (25.48 cubic meters per minute), discussed above. The media area is said to be 431 square feet (40.04 square meters).

Camfil Farr 2000^{™} Model Nos. FA 1565-02-01 and FA 1560-02-01, which are ULPA filters said to provide 99.999% efficiency at 0.3 microns and 99.99% efficiency at 0.1 microns, may be used, as well. The dimensions and resistance at airflow of these models and the models described above are the same. The FA 1565-02-01, which has the same media area as the FA 1565-01-01 discussed above, has airflow of 693 CFM (19.62 cubic meters per minute). The FA 1565-02-01, which has the same media area as the FA 1560-01-01, has airflow of 1848 CFM (52.33 cubic meters per minute).

Another example of a V-bank high efficiency filter is the Flanders Model SF2K-5-G2-CG available from Total Filtration Solutions Inc., Grand Island, NY.

The UV lamps 54 create a UV killing grid for bacterial by the glowing effect of the glass fibers and the UV light. downstream of the filter 12 are shown in FIG. 3. Preferably, the UV lamps 54 are positioned to completely and continuously illuminate the mesh surfaces of the downstream side 12b of the filter 12, respectively, during operation. The UV lamps, 54 are preferably located at least partially within the downstream facing chambers 12e defined by the transverse intersecting walls 12c of the V-bank filter 12.

The Center UV lamps 54 may also be ozone generating lamps. The air flow 48 pushes the ozone 58 behind the filter on the downstream side equally missing it with the air stream in the fan and motor operation, increasing the germicidal effect. The entire device 10 may then become a germicidal killing zone through its entire depth. Additionally, ozone facilitates the breakdown of odorants and some toxic gases, further decontaminating the air passing through the unit 10. An example of an acceptable ozone generating UV lamp is a Model GX018T5L/Ultra-V manufactured by Perkin Elmer Optoelectronics, Salem, Mass. 01970 USA.

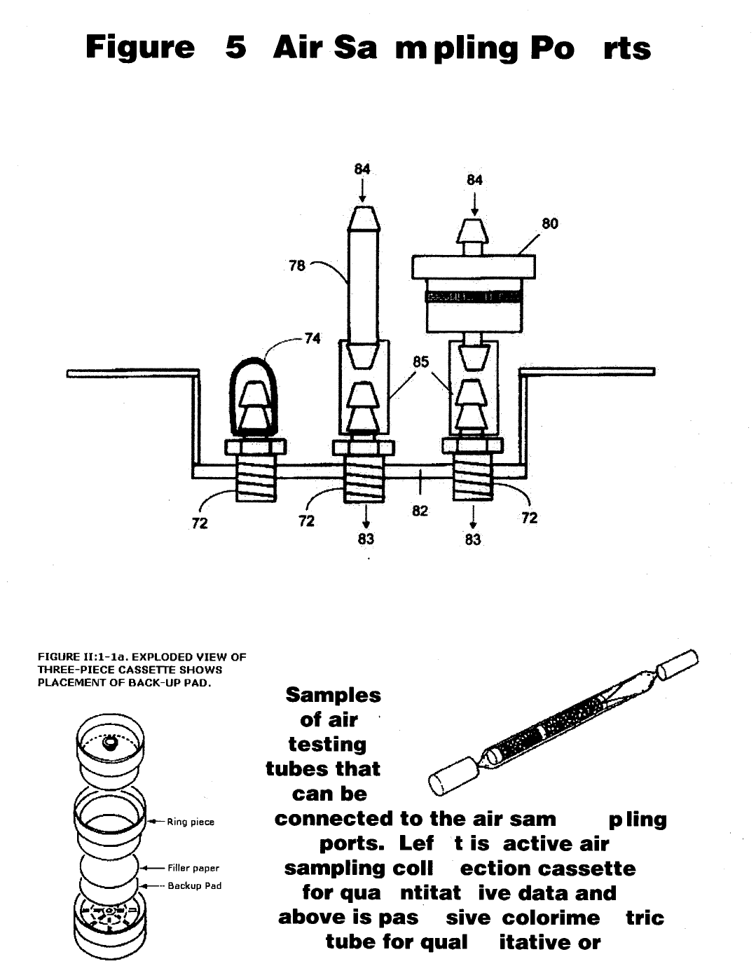

Alternatively, the ozone generator need not be a UV lamp 54. Many types of ozone generators, such as corona wires, are known and readily available. One or more ozone generators 59 may be fixed to the filter case 36 of the filter 12 or to the housing 14 of the decontamination unit 10, upstream of the filter 12, so that the filter 12 is saturated with germicidal concentrations of ozone during operation, as shown in FIG. 5. It is preferred that the ozone generator 59 be downstream of the filter 12, as not to degrade the filter and its housing and seals prematurely.

Optimal placement of a UV lamp 54 and ozone generator and/or 59 to provide a germicidal effect on and within the illuminated filter 12 requires knowledge of the UV light intensity of the lamps 54 and rate of ozone production by the ozone generator. The following equations provide guidance for calculating the germicidal effect of UV lamps and ozone generators at a given distance.

A surviving microbial population exposed to UV irradiation at wavelength o 254 nanometers (“nm”) is described by the characteristic logarithmic decay equation:

In[S(t)]=−KᵤᵥIᵤᵥt

where kᵤᵥ=standard decay-rate constant, (cm²/microW-s)

Iᵤᵥ=Intensity of UV irradiation, (microW/cm²)

t=time of exposure, (sec)

The standard decay rate constant k defines the sensitivity of a microorganism to ultraviolet irradiation. This constant is unique to each microbial species. The following table demonstrates the effect of ultraviolet irradiation on survival of selected microbes.

TABLE I

| | | Percent | Intensity | Time |
|---|---|---|---|---|
| Organism | Group | Reduction | (microW/cm²) | (sec) |
| Vaccinia | Virus | 99% | 25 | 0.02 |
| Influenza A | Virus | 99% | 25 | 0.02 |
| Coxsackievirus | Virus | 99$ | 25 | 0.08 |
| Staphylococcus | Bacteria | 99% | 25 | 1.5 |
| aureus | | | | |
| Mycobacterium | Bacteria | 99% | 25 | 1.9 |
| tuberculosis | | | | |
| Bacillus anthraci | Bacteria | 99% | 25 | 3.6 |

A surviving microbial population exposed to ozone is described by the characteristic logarithmic decay equation:

In[S(t)]=−K_{O3}I_{O3}t

where k_{O3}=standard decay-rate constant, (I/mg-s)

I_{O3}=Concentration of Ozone, (mg/I)

t=time of exposure, (sec)

The standard decay rate constant k defines the sensitivity of a microorganism to ozone. As in the use of ultraviolet irradiation, the ozone survival constant is unique to each microbial species. The following table demonstrates the effect of ozone on survival of selected microbes.

TABLE II

| | | Percent | Concentration | Time |
|---|---|---|---|---|
| Organism | Group | Reduction | (mg/l) | (sec) |
| Poliomyetis virus | Virus | <99.99% | 0.3-0.4 | 180-240 |
| Echo Virus 29 | Virus | <99.99% | 1 | 60 |
| Streptococcus sp | Bacteria | <99% | 0.2 | 30 |
| Bacillus sp | Bacteria | <99% | 0.2 | 30 |

Germicidal concentrations of ozone at a given distance from an ozone generator 54 can be determined and the ozone generator 54 can be positioned within that distance from the filter 18. To verify the location of the ozone generator 54, the concentration of ozone at the surface of the filter 12 can be measured by ozone detectors. The multispeed blower 32 can be set for air flow rates adequate to saturate the filter 12 with germicidal levels of ozone while still providing a high CFM of air flow for rapid turn over rates of air in the area being decontaminated. A preferred range is from about 600 to about 2000 CFM (16.99-67.96 cubic meters per minute).

Embodiments of the invention that include ozone generators 59 may also have UV lamps 54 downstream of the filter 12 that produce UV radiation 55 at wavelengths that facilitate the breakdown of ozone. Ultraviolet radiation in the UV “C” spectrum may be used. 255.3 nanometers is an effective wavelength, to break down ozone, for example. Accordingly, sufficient ozone can be produced at germicidal concentrations within the filter 12 while OSHA acceptable levels of ozone (less than 0.1 ppm) are released with the purified air through the outlet 30.

It may also be desirable to flood a contaminated room or space, which would typically have been evacuated, with ozone for further decontamination and odor reduction. Ozone generators 54 and/or one or more additional ozone generators 59 supported in the housing along the air path A may be used to produce ozone that is exhausted from the unit 10 through the outlet 30, into the room or space. In this case, if the UV lamps 54 emit radiation in a range that would break down ozone, they would not be turned on._The UV lamps 54 that break down ozone may be controlled by a separate switch or other such manual control device than that controlling the UV lamps, so that operation of the UV lamps 54 may be separately controlled.

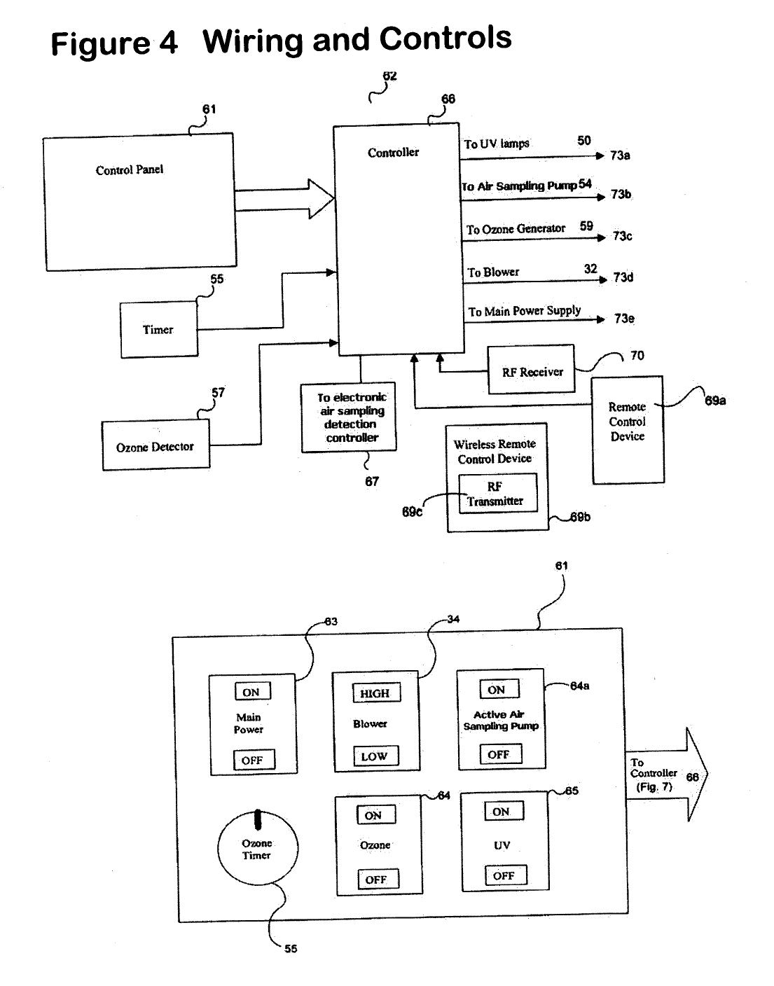

Additionally, an ozone detector 57 may be provided on the unit 10 monitor ozone levels in the air. The ozone detector 57 may be supported on the exterior of the housing 14, proximate to the air inlet 28, for example. The ozone detector 57 may be coupled to a control circuit, discussed below with respect to FIG. 4, that turns off power to the ozone generator 54 if the ozone level exceeds a predetermined level. If the unit 10 releases purified air and trace ozone in occupied areas, the preferred ozone level for shut off is the OSHA accepted level of 0.1 ppm ozone. The most preferred level for triggering shut off of ozone generation is 0.05 ppm ozone, especially if the unit is used in a hospital environment. The ozone detector 57 could also be used to maintain a desired level of ozone in a room or area. For example, if the ozone level detected by ozone detector 57 drops below a desired level, power to the ozone generator 54 and/or 59 could be turned on again

A timer 55 may also be provided to set the amount of time the ozone generators 54 and/or 59 operate. The timer 55 is shown schematically in FIG. 4.

FIG. 4 is a schematic diagram of an example of a control panel 61 that may be used to operate the decontamination unit 10 and FIG. 7 an example of a control circuit 62 for controlling operation of the decontamination unit 10. Manually operated control devices 34, 63, 64, and 65, which may be push buttons, switches or dials, for example, are provided to control the blower 32, main power to the unit 10, the ozone generators 59 and the UV lamps 54, respectively. The separate control devices 34, 63, 64 and 65 may be coupled to a controller 66, which may be a processor, such as a microprocessor, or a relay board, for example, as shown in FIG. 4. If the controller 66 is a microprocessor, memory 67 may be provided to store a program to control operation of the decontamination unit 10, based, at least in part, on inputs provided by the control devices and other optional inputs, discussed below. If the controller 66 is a relay board, the relay board acts as an interface between the control devices in the control panel 61 and the other optional inputs discussed below, and the respective components of the decontamination unit 10 being controlled. Separate control devices may be provided in the control panel 61 for the UV lamps 54, as well.

The optional inputs may include timer 55 and/or the ozone detector 57, if provided, as shown in FIG. 4. The controller 66 has outputs 73a, 73b, 73c, 73d, 73e to the UV lamps, the ozone generator 59, the blower 32, and the main power supply (not shown), respectively. Detectors which sample for gases, particulate or mists could also remotely trigger the unit operation or the manual or automatic changing of treatment modules and options.

The controls on the decontamination unit 10 may also be remotely controlled. For example, an operator may have the option to control operation of the decontamination unit 10 with a remote control device 69a, which may be a hand held control device or a computer terminal, for example, that is coupled electrically via wires to a controller 66. A wireless remote control device 69b may also be used. The wireless remote control device 69b may include a radio frequency (“rf”) transmitter 69c and an rf receiver 70 may be coupled to the controller 66. Either option enables an operator to control operation of the decontamination unit 10 from another, safe room or other location. If a remote control is not provided, the length of time of operation of the decontamination unit 10, the length of time that ozone is generated, and a delay to the start of operation, for example, may be set or programmed to provide time for the operator to leave the vicinity of the unit 10.

Decontamination of any element of the decontamination unit 10 can be done by leaving the UV lights and Ozone generator on with the blower off to create internal ozone sterilization. Connecting to the access port 7 with a vacuum to evacuate ozone, spores, and other hazards that may be within the filter housing allows the operator to open the main section of the unit while it is under negative pressure limiting any escape of contaminates from the filter section. Alternatively, this port can be used to inject gas such as Ethel oxide, chlorine dioxide and others to insure all biological or toxic materials are inert before changing the filter. For radioactive particles and other solid hazards such as asbestos, mercury and lead dust, the operator can attach a glove bag to the access maintenance door and then create negative pressure by way of the purge port 7 keeping the environment and the occupant free from escaping contamination.

The decontamination unit 10 may have a prefilter 60 attached to the housing 14 upstream of the main high volume, high capacity HEPA or HEGA filter section UV. The prefilter 60 may remove gases. It may also provide an initial filtration of larger particles, for example, facilitating subsequent filtration and sterilization by the filter 12. The prefilters may be supported in a sleeve 42 framing the air inlet 28 or may be fixed within the housing downstream of the air inlet. Choice of the prefilter 60 may depend upon the type(s) of contaminants in the air.

The prefilter may comprise activated carbon, which has a large surface area and tiny pores that capture and retain gases and odors. Activated carbon filters are readily commercially available. Activated carbon filters may be obtained from Fedders Corporation, Liberty Corner, NJ, for example.

Another commercially available prefilter that may be used may comprise zeolite, which is a three dimensional, microporous, crystalline solid with well defined structures that contain aluminum, silicon and oxygen in their regular framework. The zeolite is thermally bonded to a polyester to form the filter medium. Volatile organic compounds and gases become trapped in the void porous cavities. Zeolite is especially useful in removing ammonia and ammonium compound odors such as pet odors and urine.

Other commercially available prefilters and prefilter materials include BioSponge, PurePleat 40, MicroSponge Air Filters (TM), and electrostatic filters, for example. Additional types of prefilters are well known in the art and readily available, as well. Other suppliers of filters that may be used as prefilters include Flanders Precisionaire, St. Petersburg, Fla. and www.dustless.com, for example. The dimensions of the prefilter 60 may be 24 inches×12 inches×2 meters (length×height×depth) (0.61 meters×0.30 meters×0.05 meters), for example.

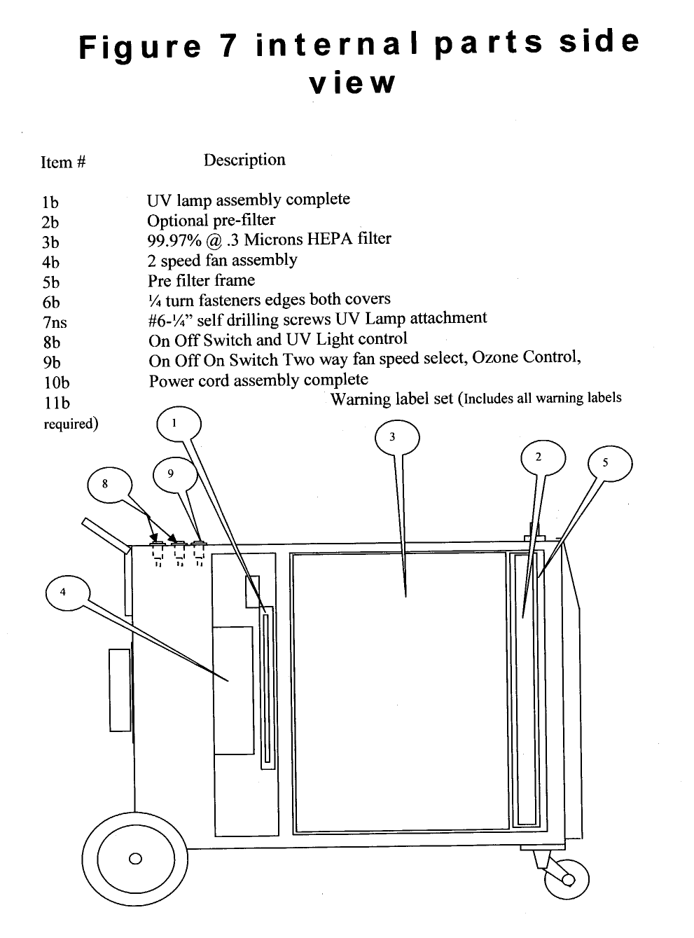

In accordance with another embodiment of the invention, a separate High Efficiency Gas Absorber (“HEGA”) module 71 may be coupled to the decontamination unit 10 as a prefilter, as shown in FIG. 8. The HEGA module 71 may be used as a gas phase scavenger to absorb nuclear, biological, or chemical (NBC) gases, for example. The HEGA module 71 has an air inlet 71a and an air outlet 71b. The air outlet 71b can be coupled to the duct adapter 68 of the decontamination unit 10, so that operation of the blower 32 will pull air into the air inlet 71a of the HEGA module 71, through the HEGA module 71, out of the air outlet 71b of the HEGA module 71 and into the air inlet 28 of the decontamination unit 10. Optionally, a duct 71c can be placed between the duct adaptor 68 of the decontamination unit 10 and the second air outlet 71b of the HEGA module 71. HEGA modules are particularly effective prefilters of gaseous contaminants. A HEGA module 71 may also be attached to the outlet duct adapter 86, in addition to or instead of attaching a HEGA module to the inlet duct adapter 68, to absorb gases that may have penetrated through the decontamination unit 10.

An example of a HEGA filter that may be used is a RS12 filled with AZM/TEDA for Warfare/Nuclear Carbon, available from Riley Equipment Co, Houston, Tex. AZM/TEDA is a composition of activated tetra-charcoal and additives dependent on the particular contaminant of concern, which is also provided by Riley Equipment Co. HEGA filters may also be obtained from Fedders Corporation, Liberty Corner, NJ, for example.

Other modules can be attached in the same manner as the HEGA unit described above which can include but are not limited to: Thermal Heat or Cold Treatment, Absorption materials such as clay, charcoal, and combinations of organic, natural or synthetic fibers, The same add on module with a chemical, or liquid mixture to neutralize toxic materials can be used in the same manner as the HEGA module working with mists, membranes, cyclonic mixing and other methods. Finally, a mixing chamber allowing the mixing of treatment or neutralizing gases can be connected in a caboose wagon train setup allowing the manual selection of the best control technology and using the particulate, and bio hazard treated high volume fan as a pump for this chain of intelligent air treatment options.

One or more air sampling ports 72 may be provided through the wall of housing 14 of the decontamination unit 10, to enable sampling of the air being drawn through the unit 10 to identify contaminants and to determine if contamination levels have been sufficiently reduced, as shown in FIGS. 1 and 2. FIG. 5 is a partial cross-sectional view of a portion of the housing 14, showing the air sampling ports 72 in more detail. The ports 72, which may have open ends, may be provided with a rubber cap 74 to close the port when not in use. An air sampling tube 78 and/or a particulate collector 80 may be inserted into a sampling port 72, as shown in FIG. 5. The ports 72 are designed to receive standard sampling tubes 78 and standard particulate collectors 80. An adapter 85 may be attached to the port 72, to receive the sampling tube 78 or particulate collector 80, after removal of the cap 74. Two of the external ports are connected by tubes to a small electric air sampling vacuum pump allowing for active air sampling at 10 to 15 LPM externally. One on these ports can have three foot long plastic tubing attached to run to the front of the unit to sample the intake or raw contaminated air. The second active port samples the operator exposure area and another internal port can sample the output air or a third external port can be added to the active sampling chain with tubing to sample all areas. This active sampling allows for faster sampling times for both Qualitative (Identification) and Quantitative (amount or concentration) results in real time analysis. This allows the operator the change and adjust prefilters, other modules and ozone UV selections to achieve the most effective chain of control technologies.

Preferably, a series of air sampling ports 72 span the housing so that an operator of the decontamination unit 10 can simultaneously test for multiple hazardous gases and particulates. During operation of the decontamination unit 10, the vacuum 83 created by the blower 32, causes air 84 exterior to the unit 10 to be drawn through the sampling tube 78 and particulate collector 80, into the air path A of the unit 10. The three remaining passive air sampling ports draw air dependent on the vacuum created naturally within negative pressure chamber that holds the fan. This is low flow of 10 cc to 50 cc per minute for color indication or sorbant tubes for sampling that can ID a substance by color changes in the field and not have to lose time with laboratory or mobile lab analysis

As mentioned above, the blower 32 is preferably located downstream of the filter 12 to draw air through the filter 12. A strong vacuum is thereby created downstream of the filter 12. Operation of the air sampling ports 72, which span the housing 14 downstream of the filter 12 and upstream of the air outlet 30, benefit from the stronger vacuum in this preferred configuration. The blower 32 may be upstream of the filter 12 and blow air downstream, through the filter 12 and past the air sampling ports 72, as well.

Air sampling glass tubes 78 are typically designed to detect one specific chemical. The operator typically first breaks both ends of the glass tube 78 to allow air to flow through the tube, and then inserts the tube into an open end of the adapter 84 on an air sampling port 72. There are many different types of commercially available colorimetric sampling tubes. Another type of air sampling tube is a Sorbant air sampling tube, which draws suspect material in the air into a material such as carbon. A tube with suspect contaminants may be provided to a laboratory that flushes and analyzes the contents to identify air borne contaminates.

Particulate collectors 80 sample for dusts and particulates. Quantitative assessment of contaminants in a particulate collector 80 requires calculation of the amount of drawn air. A rotameter may be used, for example, as known in the art to define the exact air flow volume to allow concentrations to be calculated in time weighted averages (TWA) to meet OSHA standards or a short term exposure limit (STEL). Concentration of contaminants at a low concentration may only be detected in concentrated samples created by drawing sufficient volumes of air through the collector and then determining the rate of flow by using the rotameter. Particulate collectors 80 use special materials that dissolve and allow the laboratory to measure the captured contaminates, as is also known in the art.

Air sampling techniques are well known and there are many types of tubes, samplers and air sampling equipment commercially available, as is known in the art. Air sampling guides are available from the Occupational Safety and Health Administration (OSHA), the Environmental Protection Agency (EPA), and the National Institute for Occupational Safety and Health (NIOSH), via the Internet, for example.

The embodiments of the decontamination unit 10 of the invention are particularly suited for use in industrial and medical contaminations, which may include chemical, biological and radiological accidents. The decontamination unit 10 of embodiments of the present invention may also be used after biological, chemical and radiological terrorist attacks. Detection of what is and also what is not present at a site of contamination is particularly important after a terrorist attack. Some biological and chemical agents and weapons may be deadly at very low concentrations. Having sampling ports 72 that assist in analyzing the air at a contaminated site may therefore be useful in determining the optimum approach to decontamination, including choice of prefilter, whether or not to use ozone, and required remediation time to achieve adequate decontamination, after terrorist attacks, as well as industrial and medical contaminations.

Adequate time for remediation is usually given in number of times the air in an area has passed through the decontamination device 10 or “air changes”. For example, nuisances like dust or pollen in a room require 2 to 4 air changes of the entire volume of air in the room. Typically, the more deadly the contaminant, the more air changes are required. Toxins, including but not limited to asbestos, certain gases, and most infectious material, may require 4-8 air changes. Extremely dangerous or deadly agents, such as smallpox, anthrax, chlorine dioxide, for example, may require 8-12 air changes or more depending on concentration, air flow, materials and temperature humidity conditions that affect each toxin.

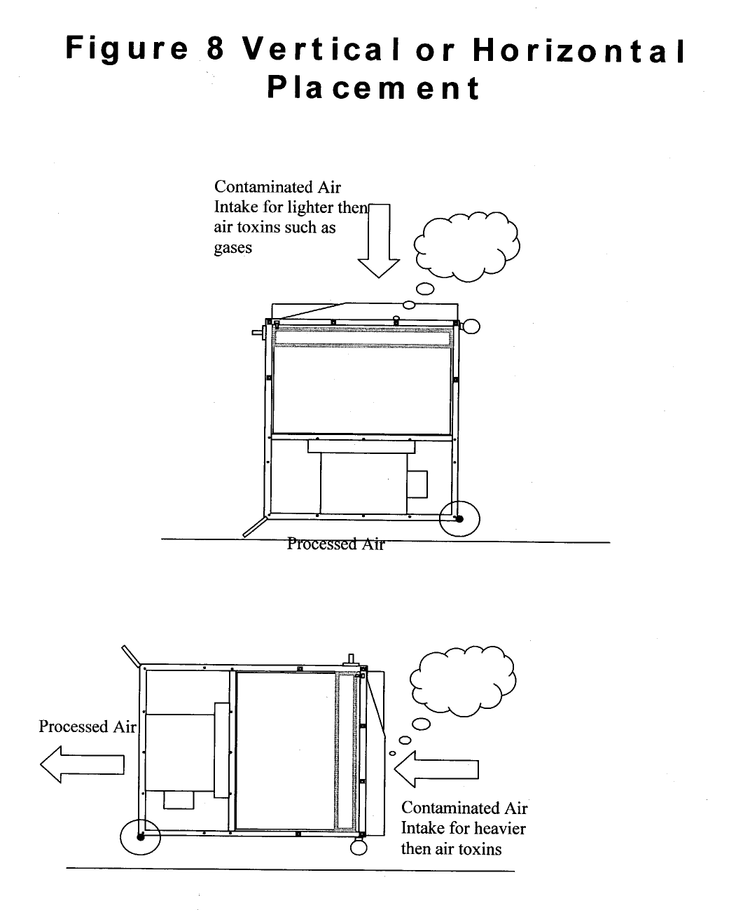

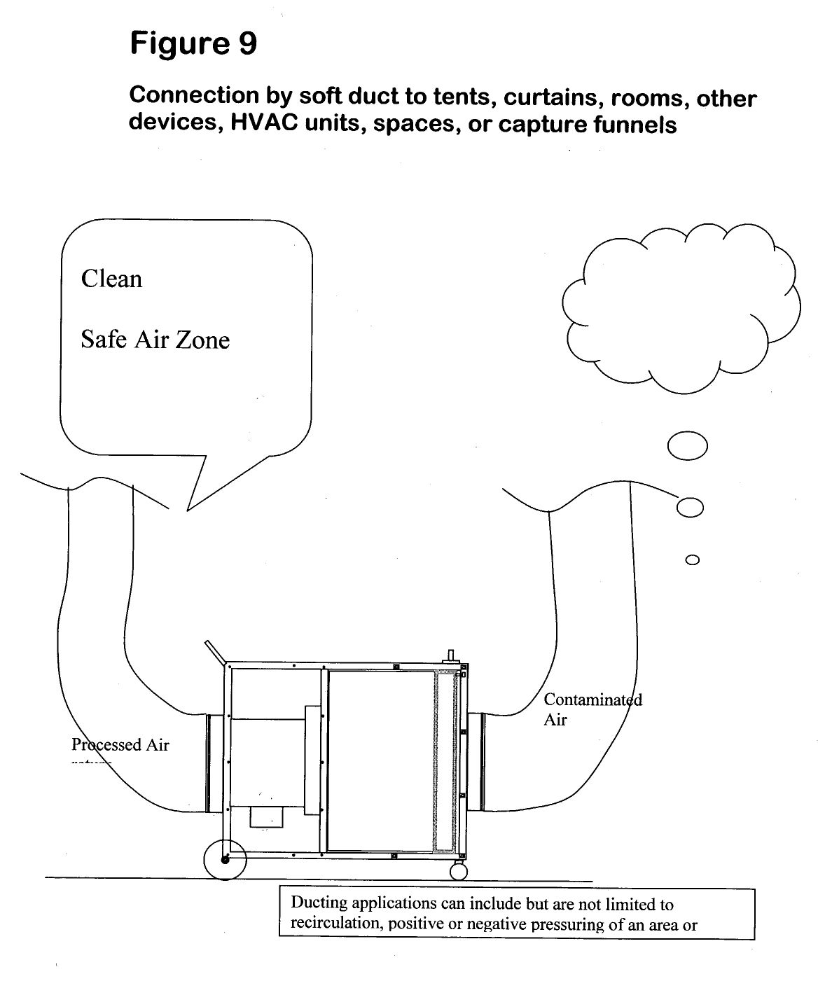

The decontamination unit 10 may also attached to ducts, FIG. 9 for connection to a room to be decontaminated, for example. Duct adapters 68 and 86 may be attached to the outside surface of the housing of unit 10, framing the air inlet 28 and air outlet 30, respectively, as shown in FIG. 10. Ducts 88 and 90 are attached to the decontamination unit 10 via the duct adapters 68, 86. Preferably, the duct adapters 68, 86 provide an air tight seal between the decontamination unit 10 and the ducts 88 and 90, respectively. These ducts can be attached to HVAC units, Decons, and other kinds of building and functional space equipment.

Contaminated air may be drawn into the unit 10 through a duct 88 and purified air or ozone laden air may be exhausted from unit 10 through duct 90. The use of ducts 88 and 90 allow for operation of the decontamination unit 10 without exposure of the operator of the unit to the contaminates in the air or the ozone being generated. Use of the decontamination unit 10 to decontaminate rooms is discussed in more detail, below.

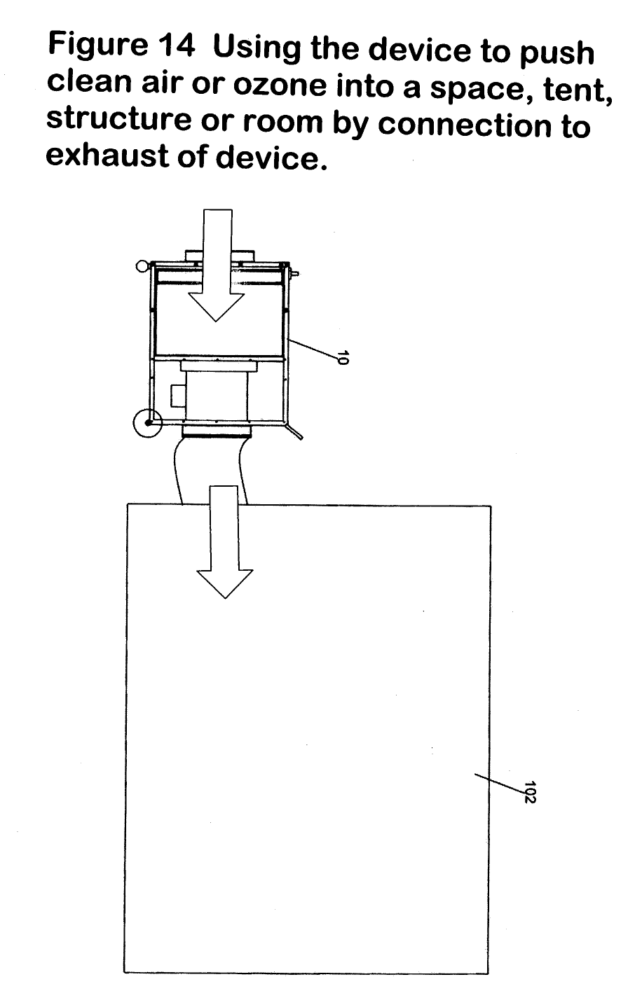

Preventing contaminated air from flowing into a room is essential in “clean rooms” for manufacturing delicate devices such as silica chips or for the creation of non-contaminated zones where people can be safe while decontamination is proceeding nearby. Operation of the decontamination unit 10 as shown in FIG. 14 creates a room or defined space that is essentially free of contaminated air. The decontamination unit 10 purifies contaminated air and continually pushes the purified air into a defined space 102 such that the pressure in the defined space, such as a room or hallway, increases. Because the air pressure in the defined space 102 is greater than the air pressure in its surroundings, air only flows out of the defined space 102. Accordingly, essentially no contaminated air can flow into the defined space 102.

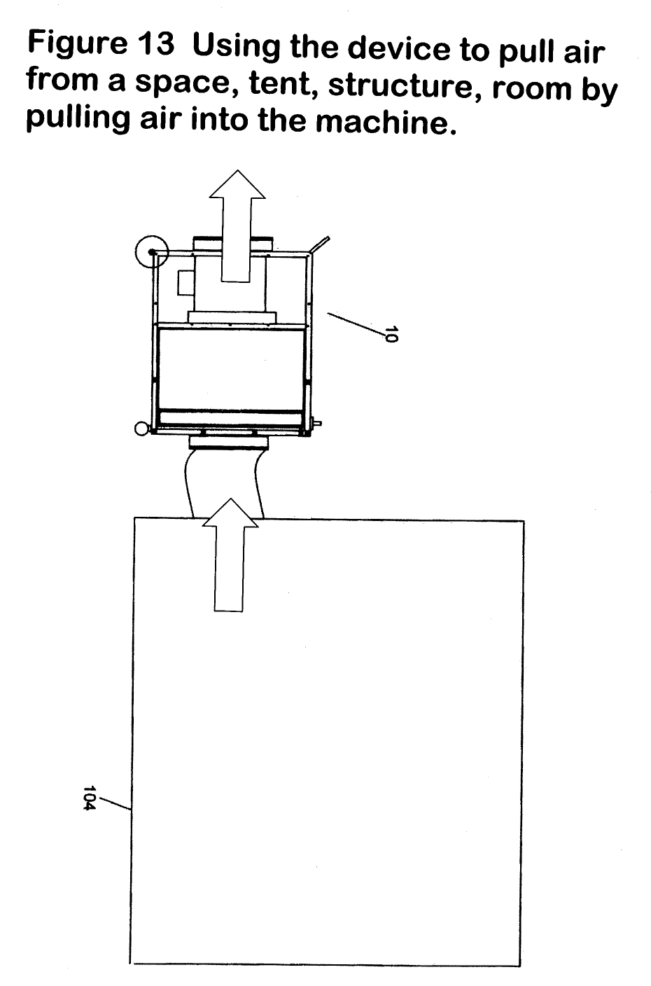

When a contaminant is localized to a room or defined space, preventing the spread of the contaminant is essential during decontamination. If the air pressure in the contaminated room is maintained at a level lower than the air pressure outside of the room, air will only flow into the contaminated room and contaminated air will not flow out of the room. Operation of the decontamination unit 10 under negative pressure is shown in FIG. 13 In FIG. 13 the decontamination unit 10 continually pulls contaminated air out of a defined space 104 such that the pressure in the defined space, such as a room or hallway, decreases. Because the air pressure in the defined space 104 is less than the air pressure in its surroundings, cleaner air flows from the surroundings into the contaminated space 104. The only contaminated air that can flow out of the contaminated space must go through the decontamination unit 10, which purifies the contaminated air.

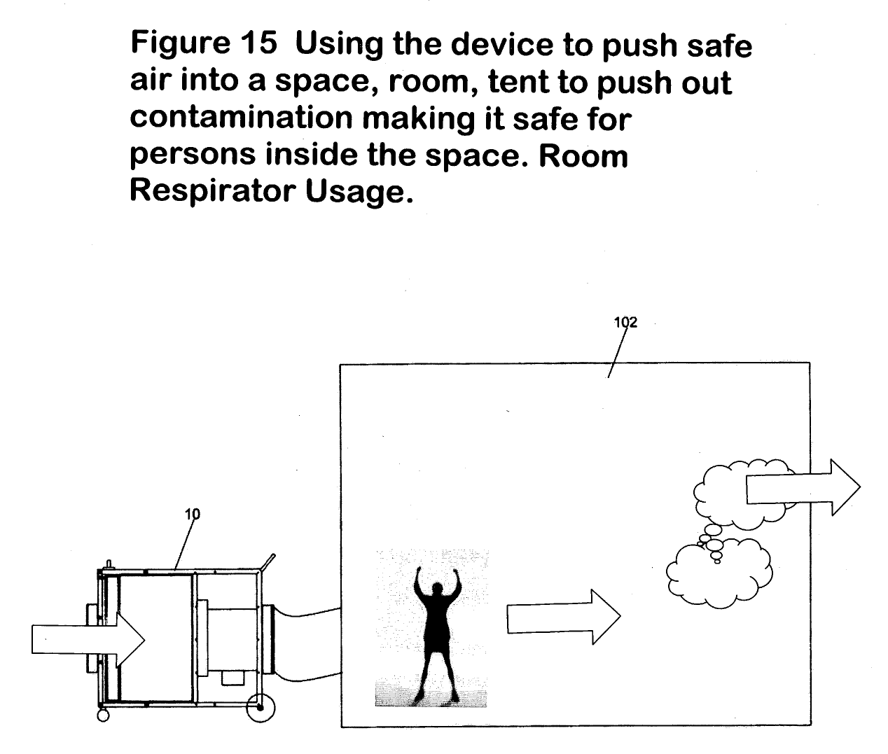

FIG. 15 shows an emergency use of the decontamination unit 105 designed for use where persons are trapped in a space where leaving the space is not possible or would create greater risk. As in the Tokyo Saran Gas attack a closed area can concentrate a deadly toxic agent and in this case an emergency responder can either position the unit as close as possible to grasp the source spread from further entry into the functional space or use the device as a fire house of clean air to push away the toxic material which is diluted at the same time. Ideally a second unit is placed down stream of the victims pulling the toxins away from people while another unit pushes clean air towards the exposed victims similar to a room sized respirator operation on a positive flow mode as in SCBA for fire departments. The effective use of several of these mobile units for such airborne hazards has been proven with smoke ejectors at fire scenes where “push/pull” ventilation pulls smoke and hot gasses away from victims and fire fighters while controlling the venting process to reduce heat and risk. The decontamination unit expands upon the concepts and principles of smoke ejector technology into the modern era where the problem is not just smoke, but chemicals, odors, weapons of mass destruction, chemical and biological and a host of other airborne hazards.

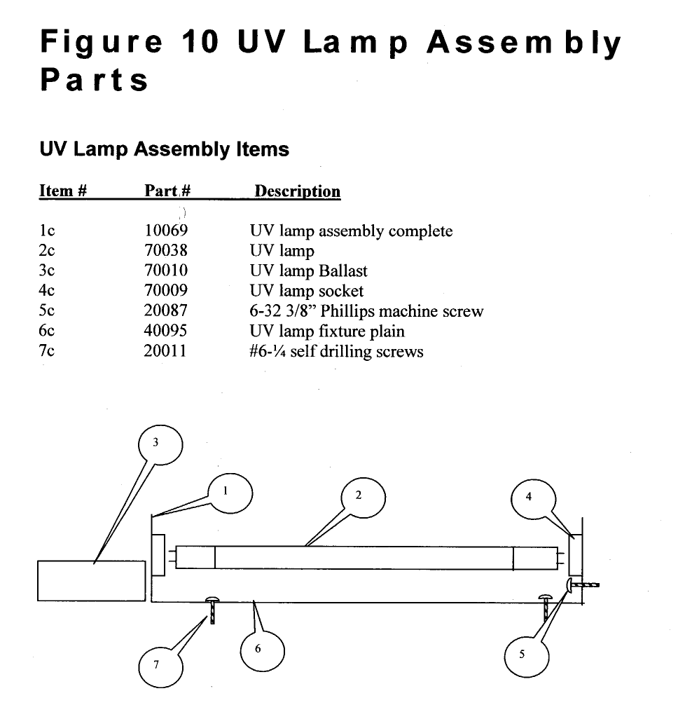

Another embodiment of the decontamination unit 10 is shown in FIG. 11, wherein the decontamination unit 10 includes two isolation or directional barriers 92 and 94 attached to the side 24 of the decontamination unit 10 containing the air inlet 28, to contain local contamination, for example. Preferably, the barriers have a light weight first frame 96 and second frame 98 attached to the top of side 24. A first wall 100 hangs from first frame 96 and a second wall (not shown) hangs from second frame 98. The isolation barriers 92, 94, combined with the side 24 of the decontamination unit 10, partially enclose a space C, to maximize flow of a contaminant into the decontamination unit 11 and minimize leakage of the contaminant to the surrounding areas. A limited chemical spill in a laboratory or hospital may be quickly contained with decontamination unit 10 by placing the isolation barriers 92, 94 around the spill. The high pressure of the blower 34 draws air, including the chemical fumes from the spill, into the unit 10, preventing dissipation of the chemical fumes away from the unit 10.

In accordance with another embodiment of the invention, aspects of the germicidal filter arrangement of the decontamination unit 10 are combined with a movable isolation device as described in U.S. Pat. No. 6,162,118 by the same inventor submitting this application.

What is claimed is:

1. A decontamination device comprising:
A housing defining an air inlet, an air outlet and a path for air to flow from the inlet to the outlet;
A stationary filter positioned within the housing, along the path, the filter having an upstream side to receive air flowing along the path and a downstream side for the exit of air from the filter, to the path;
At least one first stationary ultraviolet lamp positioned to directly illuminate the filter;
At least one second stationary ultraviolet lamp positioned to directly illuminate the downstream side of the filter; and an ozone generator proximate the filter.

2. The decontamination unit of claim 1, further comprising:
A blower within the housing, along the path, to cause air to flow along the path during operation.

3. The decontamination unit of claim 1, wherein the first and second ultraviolet lamps completely illuminate the downstream sides of the filter, respectively.

4. The decontamination device of claim 4, further comprising:
a manually operated control device supported on an exterior surface of the housing;
the control device being coupled to the ozone generator to control operation of the ozone generator.

5. The decontamination device of claim 4, further comprising:
a timer coupled to the second ozone generator to control operation of the second ozone generator.

6. The decontamination unit of claim 1, further comprising:
a manually operated control device supported on an exterior surface of the housing;
the control device being coupled to the ozone generator to control operation of the ozone generator.

7. The decontamination device of claim 1, further comprising at least one or more prefilter positioned along the path, upstream of the first ultraviolet lamp, such that air flows through the at least one prefilter prior to flowing through the filter, during operation.

8. The decontamination device of claim 1, wherein the ozone generator is an ultraviolet lamp.

9. The decontamination device of claim 1, further comprising:
at least one reflector positioned to reflect light from at least one ultraviolet lamp onto a surface of the filter.

10. The decontamination device of claim 1, wherein:
the housing has an external wall defining at least one air sampling port through the wall, to provide communication from an exterior of the housing to the path and comprising of:
At least two Passive Air Sampling Ports
At least two Active Air Sampling Ports powered by an electrical vacuum pump

11. The decontamination device of claim 1, further comprising an intake duct adapter fixed to the housing, proximate the air inlet.

12. The decontamination device of claim 1, further comprising an exhaust duct adapter fixed to the housing, proximate the air outlet.

13. The decontamination unit of claim 1, wherein the filter removes at least 99.97 percent of particles of 0.3 micron size during operation.

14. The decontamination unit of claim 16, wherein the filter removes at least 99.99 percent of particles of 0.1 micron size during operation.

15. The decontamination unit of claim 1, wherein the filter comprises ultraviolet transmissive material.

16. The decontamination unit of claim 1, wherein, the second ultraviolet lamp emits radiation capable of breaking down ozone, during operation.

17. The decontamination unit of claim 20, further comprising:
a manually operated control device supported on an exterior of the housing;
the control device being coupled to the second ultraviolet lamp to control operation of the second ultraviolet lamp.

18. The decontamination unit of claim 21, further comprising:
a manually operated control device supported on an exterior surface of the housing;
the control device being coupled to the ozone generator, to the blower and to the second UV lamp such that activation of the switch turns on the ozone generator, turns off the blower and turns off the second UV lamp.

19. The decontamination unit of claim 1, further comprising:
an ozone detector proximate to the air inlet;
the ozone detector being coupled to the ozone detector to control operation of the ozone generator.

20. The decontamination unit of claim 1, further comprising:
a high efficiency gas absorber coupled to the inlet.
A thermal treatment module.
An absorptive treatment module.
An chemical or liquid treatment module.
A gas injected treatment module.
A rapid air change adapter to allow filter changes in less than three seconds while the unit is running.
The ability to run the unit in vertical or horizontal modes standing on a back handle or stand to support the unit.
A method of decontaminating air comprising:
flowing air through a filter from an upstream side to a downstream side of the filter;
Using commercially available specialty pre filters designed for a wide range of toxic hazards, while the air is flowing through the filters;
illuminating the entire downstream side of the filter with ultraviolet light, while the air is flowing through the filter; and
permeating the filter with ozone while the air is flowing through the filter.

21. The method of wherein the method is implemented by a device, the method further comprising:
remotely controlling operation of the device.
remotely sensing sampling data to detect hazards.
operating the device in accordance with a program.

22. The method of claim 20 wherein the method is implemented by a device, the method further comprising:
Operating the device manually in a push or pull mode within functional spaces.
Connecting the decontamination unit in a chain or series with other treatment technology
The use of collars, ducts, tubes, and tunnels to connect components of different treatment modules
The active ability to change, redirect, or adjust the sequence or operational function of various modules.
The use of controls and blowers to alternative, change, adjust, reduce or increase air flow though the decontamination unit to allow different residency times for contaminated air to benefit most efficiently from selected filters and treatment options or technology.

23. A decontamination unit of claim 20 comprising:
a housing defining an inlet, an outlet, and a path for air to flow from the inlet to the outlet;
a filter positioned along the path to filter air flowing along the path, the filter comprising a plurality of transverse intersecting walls defining at least one upstream facing chamber to receive air along the path, and a downstream side for air to exit from the filter, to the path; and
at least one ultraviolet lamp upstream of the filter, facing the at least one chamber, to completely, directly illuminate the at least one chamber.

24. The decontamination unit of claim 20 further comprising:
a blower within the housing, along the path, to cause air to flow along the path during operation.

25. The decontamination unit of claim 20, further comprising:
at least one reflector upstream of the at least one ultraviolet lamp, to reflect ultraviolet light emitted by the at least one ultraviolet lamp, onto the at least chamber.

26. The decontamination unit of claim 30, wherein the at least one ultraviolet lamp is at least partially within a region defined by the chamber.

27. The decontamination unit of claim 28, wherein the downstream side of the filter defines at least one downstream facing chamber, the unit further comprising:
at least one second ultraviolet lamp downstream of the filter, facing the at least one downstream facing chamber, to completely, directly illuminate the at least one downstream facing chamber.

28. The decontamination unit of claim 32, further comprising:
at least one second reflector downstream of the at least one second ultraviolet lamp, to reflect ultraviolet light emitted by the at least one second ultraviolet lamp, onto the at least downstream facing chamber.

29. The decontamination unit of claim 33, wherein the at least one second ultraviolet lamp is within a second region defined by the downstream facing chamber.

30. The decontamination unit of claim 34, wherein:
the filter comprises a plurality of transverse, intersecting walls;
the plurality of walls define a plurality of upstream and downstream facing V-shaped chambers;

31. The method of, wherein the method is implemented by a device, the method further comprising:
Portable and mobile decontamination units that can be connected together in a chain or caboose like fashion.
The method of push or pulling air to contain the spread of toxic airborne contamination.
Air sampling ability in both passive and active modes located on the unit with the ability to same air before it enters the unit, during and after treatment.
The use of a high powered fan moving over 500 cfm of air that can be used to provide emergency breathing air or room respirator effect to multiple victims.

32. The decontamination unit of claim 28, further comprising:
an ozone generator proximate the filter.

33. The decontamination unit of claim 28, further comprising:
a prefilter along the path, upstream of the filter.

34. The decontamination unit of claim 28, wherein the filter removes at least 99.97 percent of particles of 0.3 micron size, during operation.

35. The decontamination unit of claim 28, wherein the filter comprises ultraviolet transmissive material.

36. The decontamination unit of claim 28, wherein the filter is fire resistant.

37. The decontamination unit of claim 28, wherein the housing has an external wall defining an air sampling port through the wall, enabling communication between an exterior of the housing and the path.

38. A method of decontaminating air, comprising:
flowing air through a filter, the filter having at least one upstream facing chamber to receive air to be filtered; and
completely, directly illuminating the at least one upstream facility chamber with ultraviolet light while the air is flowing through the filter.

39. The method of claim 43, wherein the filter further comprises at least one downstream facing chamber, the method further comprising:
completely, directly illuminating the at least one downstream facing chamber with ultraviolet light while the air is flowing through the filter.

40. The method of claim 44, further comprising:
permeating the filter with ozone while the air is flowing through the filter.

41. A decontamination unit comprising:
a housing defining an inlet, an outlet, and a path for air to flow from the inlet to the outlet;
a filter positioned along the path to filter air flowing along the path, the filter having an upstream side defining at least one upstream facing chamber to receive air along the path, and a downstream side for air to exit from the filter, to the path; and
at least one ultraviolet lamp upstream of the filter, positioned at least partially within a region defined by the chamber, to illuminate the chamber.

42. The decontamination unit of claim 46, further comprising:
a blower to cause air to flow along the path.

43. A method of using a decontamination unit in a mobile, movable, fashion comprising:
a housing defining an inlet, an outlet, and a path for air to flow from the inlet to the outlet; and
a filter positioned along the path, to filter air flowing along the path;
the housing having an external wall defining an air sampling port through the wall, enabling communication between an exterior of the housing and the path.
Locking front wheels
A rear handle doubling as a vertical support stand.

44. The decontamination unit of claim 1, further comprising a penetration connection though the underside of the unit sealed with a threaded cap when not in use.
The port is a cleanout connection port or injector site of cleaning or neutralizing agent's installed to allow:
Attachment of a vacuum such as a HEPA vacuum.
Attachment of suction
Attachment of a hose to inject gas or disinfectant or neutralization gas

45. The method of claim 44 wherein trapped toxins inside the filter can be made inert or contained during maintenance or filter changes.

46. The decontamination unit of claim 48, wherein:
the port is an air sampling port; and
air is drawn from the exterior of the housing, through the port, to the path.

47. The decontamination unit of claim 51, further comprising a sampling tube received within the air sampling port, to collect air external to the housing.

48. The decontamination unit of claim 51, further comprising a particulate collector received within the port, to collect particles in the air external to the housing.

49. The decontamination unit of claim 48, further comprising:
a selectable prefilter along the path, upstream of the filter.

50. The decontamination unit of claim 54, wherein the selectable filter may be selected based on air sampling results.

51. The decontamination unit of claim 48, further comprising:
Locking front wheels, a front and rear handle allowing unit to be stand in a vertical or horizontal position.
at least one second ultraviolet lamp positioned to illuminate a downstream side of the filter;
at least one ozone generator proximate the filter.

52. A method of decontaminating air with a decontamination unit, the method comprising:
flowing air along a path through the unit, the path including a filter;
filtering the air; and
collecting an air sample, via the unit.

53. The method of claim 58, wherein the decontamination unit comprises a wall defining an air sampling port providing communication between an exterior to the wall and the path, the method further comprising:
drawing air external to the unit through the air sampling port, towards the path, to collect the sample.

54. The method of claim 59, further comprising:
collecting air samples by a sampling tube received within the air sampling port.

55. The decontamination unit of claim 59, comprising collecting particles from the air with a particulate collector received within the port.

56. The method of claim 58, further comprising:
selecting a prefilter based on sampling results; and
positioning the prefilter upstream of the filter in the decontamination unit.

57. An isolation device, comprising:
a frame;
a barrier mounted on the frame to partially enclose a space;
an air conducting unit attached to the barrier, the air conducting unit having an air inlet exposed to the enclosed space and an air outlet exposed to an exterior of the device, to conduct air between the partially enclosed space and the exterior of the device; and
a recycling vent to provide communication from the air conducting unit to a location proximate the enclosed space, to provide filtered air to the enclosed space.

58. The isolation device of claim 63, further comprising:
a blower for causing air to flow through the air conducting unit from the air inlet to the air outlet, within the air conducting unit.

59. The isolation device of claim 63, further comprising:
a baffle within the air conducting unit to deflect at least a portion of the air flowing from the air inlet to the air outlet through the air conducting unit out of the recycling vent, during operation.

60. The isolation unit of claim 65, wherein from 50% to 75% of the air flowing from the air inlet to the air outlet through the air conducting unit is deflected out of the recycling vent.

61. The isolation device of claim 63, further comprising:
a filter within the air conducting unit, the filter having an upstream side to receive air flowing through the unit and a downstream side for the exit of air passing through the filter;

62. The isolation device of claim 67, further comprising an ozone generator proximate the filter.

63. The isolation device of claim 63, further comprising:
a tent wherein a bed or portion of a bed can be placed within the partially enclosed space.

64. The isolation device of claim 63, wherein the frame is mobile.

65. A method of decontaminating a room, comprising:
producing germicidal concentrations of ozone throughout the room;
causing air in the room to flow through a filter, from an upstream side of the filter to a downstream side of the filter; and
illuminating the upstream and downstream sides of the filter with germicidal levels of ultraviolet light.

66. A method of decontaminating a room, comprising:
drawing air from the room through a filter having an upstream side to receive the air and a downstream side for air to exit the filter;
illuminating an entire upstream side of the filter with ultraviolet light, while the air is flowing through the filter;
illuminating an entire downstream side of the filter with ultraviolet light, while the air is flowing through the filter;
permeating the filter with ozone while the air is flowing through the filter; and
ducting the filtered air out of the room to create a negative pressure within the room.

67. The method of claim 1 wherein the ability to rapid change pre filters without shutting down the unit or compromising the efficiency of the unit. The method comprising:
A slot or filter holder allowing for rapid prefilter changes as air is flowing through the filter.

68. A method of decontaminating a room, comprising:
flowing air outside of the room through a filter having an upstream side to receive the air and a downstream side from which the air exits the filter;
illuminating an entire downstream side of the filter with ultraviolet light, while the air is flowing through the filter;
permeating the filter with ozone while the air is flowing through the filter; and
ducting the filtered air into the room to create a positive pressure within the room.
